(19) United States
(12) Patent Application Publication
(10) Pub. No.: US 2026/0163016 A1
(43) Pub. Date: Jun. 11, 2026

(54) DATA MINING METHOD, ELECTROLYSIS DEVICE, CATALYST LAYER, ELECTROLYSIS METHOD, AND OXYGEN REDUCTION CATALYST

(71) Applicants: TOHOKU UNIVERSITY, Sendai-shi (JP); The University of Sydney, New South Wales (AU)

(72) Inventors: Hao LI, Sendai-shi (JP); Xue JIA, Sendai-shi (JP); Li WEI, New South Wales (AU)

(21) Appl. No.: 19/408,824

(22) Filed: Dec. 4, 2025

(30) Foreign Application Priority Data
Dec. 6, 2024 (JP) 2024-213859

(51) Int. Cl. H01M 4/90; G16C 20/70

(52) U.S. Cl. CPC H01M 4/9016; G16C 20/70

(57) ABSTRACT

An electrolysis device includes a first electrode, a first catalyst layer provided on the first electrode, a second electrode, a second catalyst layer provided on the second electrode, an electrolyte interposed between the first electrode and the second electrode, and a container containing the first electrode, the first catalyst layer, the second electrode, the second catalyst layer, and the electrolyte, wherein the first catalyst layer contains Sb₂WO₆.

### CROSS-REFERENCE TO RELATED APPLICATIONS

The present application is based upon and claims the right of priority to JP Patent Application No. 2024-213859, filed on Dec. 6, 2024, the disclosure of which is hereby incorporated by reference herein in its entirety for all purposes.

### BACKGROUND OF THE INVENTION

### Field of the Invention

The present invention relates to a data mining method, an electrolysis device, a catalyst layer, an electrolysis method, and an oxygen reduction catalyst.

### Description of Related Art

As a type of fuel cell, a hydrogen-oxygen secondary battery, a metal-air secondary battery and the like are known, and a positive electrode of each of these batteries contains oxygen supplied from the outside as a positive electrode active material. A negative electrode of the hydrogen-oxygen secondary battery contains hydrogen as a negative electrode active material. A negative electrode of the metal-air secondary battery contains a metal as a negative electrode active material. Charging and discharging can be performed by an electrolytic reaction on the positive electrode side. Platinum compounds are generally used as catalysts for the positive electrode in consideration of chemical stability (Patent Document 1, etc.), but there is a challenge in reducing the operating cost of fuel cells according to an electrolytic reaction using them. Inexpensive catalysts having catalytic performance equivalent to that of platinum compounds are required.

As a general method for searching for unknown metal oxides, data mining using computational material databases is known. This data mining can be used to search for metal oxides having a predetermined catalytic performance. However, the results predicted by data mining may not match the experimental results. If data mining does not consider the surface state of metal oxides, there is a possibility of the metal oxides being overlooked and incorrectly identified through such data mining. There is a need for a data mining method in which metal oxides having predetermined catalytic performance can be searched for with high accuracy.

### PATENT DOCUMENTS

[Patent Document 1] WO2018/194008

### SUMMARY OF THE INVENTION

The present invention has been made in view of the above circumstances, and an object of the present invention is to provide an electrolysis device, an electrolysis method, and a catalyst layer (oxygen reduction catalyst) used in electrolysis, which use an inexpensive metal oxide having excellent catalytic performance, and a data mining method that enables searching for a metal oxide among a plurality of materials.

In order to achieve the above object, the present invention provides the following aspects.
(1) An oxygen reduction catalyst according to one aspect of the present invention includes Sb₂WO₆.
(2) An electrolysis device according to one aspect of the present invention includes a first electrode, a first catalyst layer provided on the first electrode, a second electrode, a second catalyst layer provided on the second electrode, an electrolyte interposed between the first electrode and the second electrode, and a container containing the first electrode, the first catalyst layer, the second electrode, the second catalyst layer, and the electrolyte, wherein the first catalyst layer contains Sb₂WO₆.
(3) A catalyst layer according to one aspect of the present invention is a catalyst layer that is provided in an electrode constituting an electrolysis device and promotes an electrolytic reaction, including Sb₂WO₆.
(4) An electrolysis method according to one aspect of the present invention includes a process of generating a potential difference between two electrodes with an electrolyte therebetween; a process of supplying oxygen molecules to a first electrode on the high potential side between the electrodes; and a process of supplying a reducing agent for the oxygen molecules to a second electrode on the low potential side between the electrodes, wherein a catalyst layer containing Sb₂WO₆ is provided on the surface of the first electrode.
(5) In the electrolysis method according to (4), preferably, an oxygen reduction reaction is caused in the catalyst layer to generate water.
(6) In the electrolysis method according to (4) or (5), hydrogen molecules or hydrogen compound molecules may be used as the reducing agent.
(7) In the electrolysis method according to any one of (4) to (6), preferably, the pH of the electrolyte around the catalyst layer and the potential of the first electrode are adjusted so that the values are included in a region between a first line segment and a second line segment in a Pourbaix diagram, the first line segment being a line segment connecting a point at which the pH is 0 and the potential is 0.6 V and a point at which the pH is 14 and the potential is −0.226 V, and the second line segment being a line segment connecting a point at which the pH is 0 and the potential is 1 V and a point at which the pH is 14 and the potential is 0.174 V.
(8) In the electrolysis method according to any one of (4) to (7), the pH around the catalyst layer may be 1.3 or more and 12.6 or less.
(9) A data mining method according to one aspect of the present invention is a data mining method of searching for a metal oxide as a catalyst in an oxygen reduction reaction, including a first process of selecting a predetermined metal oxide from metal oxides recorded in a database; a second process of selecting a metal oxide that satisfies the condition that Eₕᵤₗₗ is 0 from the metal oxides selected in the first process; a third process of selecting a metal oxide that satisfies the condition that E_{Form} is minimized from the metal oxides selected in the second process; a fourth process of creating a Pourbaix diagram for each metal oxide selected in the third process, with a stable region being a pH and potential region in which the Gibbs free energy is 0.5 eV·atom⁻¹ or less; and a fifth process of selecting, as an electrode catalyst, a metal oxide that provides a Pourbaix diagram indicating the presence of a solid phase in a predetermined reaction among the Pourbaix diagrams created in the fourth process.
(10) In the data mining method according to (9), combinations of the metal oxides selected in the third process and the Pourbaix diagrams created in the fourth process may be recorded in a predetermined recording device, and the metal oxide to be used as the electrode catalyst is selected by searching records in the recording device.
(11) The data mining method according to (9) or (10) may further include a process of selecting one containing a predetermined number of metal elements from the metal oxides selected in the third process.

According to the present invention, it is possible to provide an electrolysis device, an electrolysis method, and a catalyst layer (oxygen reduction catalyst) used in electrolysis, which use an inexpensive metal oxide having excellent catalytic performance, and a data mining method that enables searching for a metal oxide among a plurality of materials.

### BRIEF DESCRIPTION OF THE DRAWINGS

FIG. 1 is a configuration diagram of a fuel cell used in an electrolysis method according to one embodiment of the present invention.

FIG. 2 is a process flow of a data mining method according to one embodiment of the present invention.

FIG. 3 is a graph showing the numbers of metal oxides selected in example processes.

FIG. 4 is a graph showing metal oxides after a third process classified according to the number of metal elements contained.

FIG. 5A is a diagram showing classified reactions in which metal elements exhibit thermodynamic stability in solutions with a pH of 0.

FIG. 5B is a diagram showing classified reactions in which metal elements exhibit thermodynamic stability in solutions with a pH of 7.

FIG. 5C is a diagram showing classified reactions in which metal elements exhibit thermodynamic stability in solutions with a pH of 14.

FIG. 6 is a graph showing the results of X-ray diffraction measurement performed on Sb₂WO₆ synthesized in examples.

FIG. 7 shows an image of Sb₂WO₆ obtained using a scanning electron microscope.

FIG. 8 shows images of the results of elemental mapping of Sb₂WO₆ by energy dispersive X-ray spectroscopy.

FIG. 9 shows an image of Sb₂WO₆ obtained by ICP-OES measurement.

FIG. 10A is the spectrum obtained by performing X-ray photoelectron spectroscopy measurement on Sb₂WO₆.

FIG. 10B is an enlarged view of a part of the spectrum of FIG. 10A.

FIG. 10C is an enlarged view of an another part of the spectrum of FIG. 10A.

FIG. 11 is a graph showing the LSV curves measured by a rotating ring-disk electrode method.

FIG. 12 is a graph showing the number of electrons contributing to an oxygen reduction reaction measured by a rotating ring-disk electrode method.

FIG. 13 is a graph showing the LSV curves measured by the rotating ring-disk electrode method.

FIG. 14 is a graph showing the results of chronoamperometry measurement performed on Sb₂WO₆ in an acidic solution and an alkaline solution.

FIG. 15 is a graph showing the results obtained by correcting the X-ray diffraction measurement results in FIG. 6 using a density functional theory.

FIG. 16A is a graph showing the results obtained by correcting the X-ray photoelectron spectroscopy measurement results in FIG. 10B using a density functional theory.

FIG. 16B is a graph showing the results obtained by correcting the X-ray photoelectron spectroscopy measurement results in FIG. 10C using a density functional theory.

FIG. 17A shows a TEM image (top) of Sb₂WO₆ in an acidic electrolytic solution and an EDS elemental mapping image (bottom).

FIG. 17B shows a TEM image (top) of Sb₂WO₆ in an alkaline electrolytic solution and an EDS elemental mapping image (bottom).

FIG. 18 is a Pourbaix diagram showing the surface state of a catalyst layer.

FIG. 19 is a graph showing the relationship between the binding energy of OH⁻ with respect to the catalyst layer and the current density according to oxygen reduction.

### DETAILED DESCRIPTION OF THE INVENTION

Hereinafter, an electrolysis method and a data mining method according to embodiments to which the present invention is applied will be described in detail with reference to the drawings. Here, in the drawings used in the following description, in order to facilitate understanding of features, feature parts are enlarged for convenience of illustration in some cases, and dimensional ratios of components are not necessarily the same as actual ones. In addition, materials, sizes, and the like provided as exemplary examples in the following description are merely examples, and the present invention is not limited thereto, and they can be appropriately changed and implemented within a range not changing the scope and spirit of the invention.

### Electrolysis Device

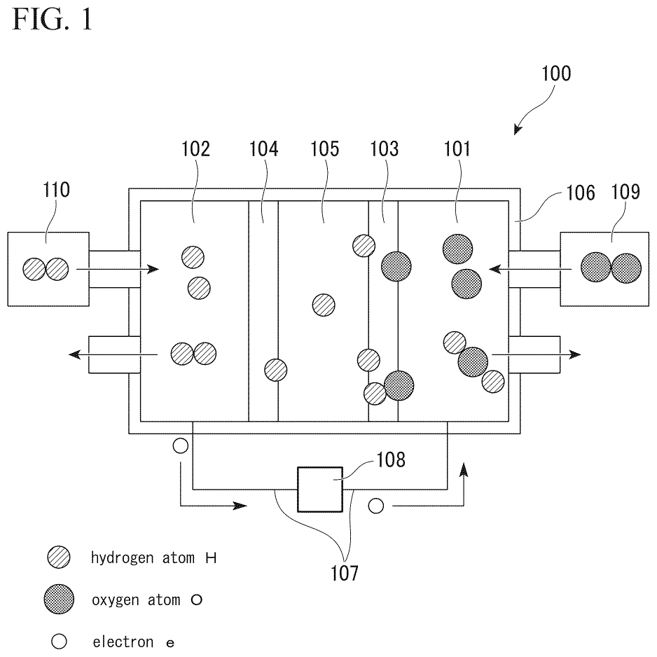

FIG. 1 is a diagram schematically showing the configuration of a fuel cell 100 as an electrolysis device used in an electrolysis method according to one embodiment of the present invention. The fuel cell 100 mainly includes two electrodes (a first electrode 101 and a second electrode 102), catalyst layers (a first catalyst layer 103 and a second catalyst layer 104) provided on the electrodes to promote an electrolytic reaction, an electrolyte 105 interposed between the two electrodes, a container 106 containing them, and a power generation unit (power generating element) 108 connected between the two electrodes through a wiring 107.

The first electrode 101 and the second electrode 102 are made of a chemically stable and conductive material (platinum, etc.). The electrolyte 105 is made of a chemically stable material (sulfuric acid, etc.) through which a reducing agent for oxygen molecules O₂ can easily propagate. The electrolyte 105 is preferably provided not only between the first electrode 101 and the second electrode 102, but also around the entire periphery of the first electrode 101 and the entire periphery of the second electrode 102.

The first catalyst layer (catalyst layer) 103 formed on the surface of the first electrode 101 contains Sb₂WO₆ (molecules) as an essential material. The first catalyst layer may contain only Sb₂WO₆ or may contain Sb₂WO₆ as a main component, preferably at 10% or more, for example, 90%. A preferable catalyst material contained in the first catalyst layer 103 can be selected using a data mining method described below. The thickness of the first catalyst layer 103 is not particularly limited. The material of the second catalyst layer 104 formed on the surface of the second electrode 102 is not particularly limited, and may be any chemically stable material having a function of promoting reduction of the material (here, hydrogen molecules H₂) supplied to the second electrode 102.

The formation of the first catalyst layer (oxygen reduction catalyst) 103 containing Sb₂WO₆ is not particularly limited, and can be performed, for example, by the following procedure. SbCl₃ and Na₂WO·2H₂O are dissolved in water and stirred. A tungstic acid solution is added to the stirred solution and stirred again. The re-stirred solution is heated at a predetermined temperature for a predetermined time, and additionally centrifuged and dried to collect an Sb₂WO₆ powder (solid). The collected powder can be applied to the surface of the first electrode 101 to form the first catalyst layer 103.

Since the oxygen reduction reaction in the first catalyst layer 103 containing Sb₂WO₆ is a four-electron reaction in which water is generated, similar to the oxygen reduction reaction in the catalyst layer containing platinum compound molecules, a larger current can pass through the power generation unit 108 compared to the case of a two-electron reaction. In addition, when chemically stable Sb₂WO₆ are used as a catalyst material, the magnitude of a current flowing through the power generation unit 108, that is, a current generated by the fuel cell, can be maintained for a long period.

### Electrolysis Method

An electrolysis method of the present embodiment is a method of reducing oxygen, and mainly includes the following first process, second process, and third process.

### First Process

A potential difference is generated between the first electrode 101 and the second electrode 102. This potential difference can be generated by applying a voltage between the first electrode 101 serving as a positive electrode and the second electrode 102 serving as a negative electrode. In this case, relatively, the first electrode 101 has a high potential, and the second electrode 102 has a low potential.

In consideration of chemical stability, it is preferable to adjust the pH of the electrolyte 105 around the first catalyst layer 103 and the potential of the oxidation-reduction reaction of the first electrode 101 so that the values are included in a region between a first line segment and a second line segment defined below in the Pourbaix diagram. The first line segment is a line segment connecting a point at which the pH is 0 and the potential is 0.6 V and a point at which the pH is 14 and the potential is −0.226 V. The second line segment is a line segment connecting a point at which the pH is 0 and the potential is 1 V and a point at which the pH is 14 and the potential is 0.174 V. Here, in order to obtain high catalytic activity, the pH around the first catalyst layer 103 is preferably 1.3 or more and 12.6 or less.

### Second Process

Oxygen molecules O₂ are supplied to the first electrode (air electrode) 101. The supply of oxygen molecules O₂ may be performed using an oxygen supply source 109 as shown in FIG. 1, or may be performed by exposing a part of the first electrode 101 to the atmosphere.

### Third Process

A reducing agent for oxygen molecules O₂ is supplied to the second electrode (fuel electrode) 102. As the reducing agent, hydrogen molecules or hydrogen compound molecules are preferably used. Here, hydrogen molecules H₂ are used as a reducing agent for oxygen molecules O₂. The supply of the reducing agent (hydrogen molecules H₂) is performed using a reducing agent supply source (hydrogen supply source) 110 as shown in FIG. 1.

A hydrogen molecule H₂ is supplied to the second electrode (fuel electrode) 102 on the low potential side and separates into two hydrogen atoms on the second electrode 102, releases electrons e⁻, and becomes hydrogen ions H⁺. The released electrons e⁻ reach the first electrode 101 through the wiring 107. An oxygen molecule O₂ supplied to the first electrode 101 on the high potential side separates into two oxygen atoms on the first electrode 101, receives the electrons e⁻, and becomes oxygen ions O²⁻.

On the other hand, the hydrogen ions H⁺ propagate through the electrolyte 105, reach the first electrode 101, and react with an oxygen ion O²⁻ on the first electrode 101, and a water molecule H₂O is generated. In this case, the reaction is a four-electron reaction represented by O₂+4H⁺+4e⁻→2H₂O, and a larger current can be generated compared to a two-electron reaction. In this manner, the supplied oxygen molecules O₂ can be reduced.

As described above, according to the electrolysis method of the present embodiment, the first catalyst layer 103 containing Sb₂WO₆ is provided on the first electrode 101 that supplies oxygen molecules O₂. Sb₂WO₆ can be synthesized more inexpensively than platinum compounds, and has a catalytic performance equivalent to that of platinum compounds. Since the oxygen reduction reaction occurring at the first electrode 101 becomes a four-electron reaction in which water is generated with Sb₂WO₆ therebetween, a large current can be extracted.

In addition, since an Sb₂WO₆ is composed of chemically stable elements, it is possible to maintain catalytic performance for a long time, and as a result, a large current can be extracted.

### Data Mining Method

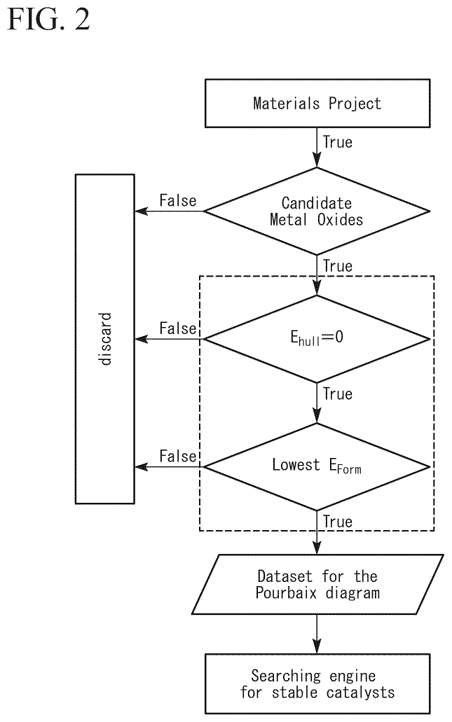

FIG. 2 is a process flow of a data mining method according to one embodiment of the present invention. The data mining method of the present embodiment is a method of searching for a metal oxide electrode catalyst, and mainly includes the following processes.

### First Process

From metal oxides recorded in the database, predetermined metal oxides are selected and other metal oxides are excluded. Examples of metal oxides to be selected include metal oxides that can be obtained at low costs, and metal oxides that do not contain a radioactive element, a halogen element, an element that is a gas at room temperature and atmospheric pressure, an organic element or the like.

### Second Process

From the metal oxides selected in the first process, metal oxides that satisfy the condition that the Energy above hull (Ehull) that is an index of relative thermodynamic stability is 0 are selected, and other metal oxides are excluded. Eₕᵤₗₗ can be determined by first principle calculation, and metal oxides with a lower Eₕᵤₗₗ are more stable and most easy to produce.

### Third Process

From the metal oxides selected in the second process, metal oxides that satisfy the condition that the formation energy E_{Form} is minimized are selected, and other metal oxides are excluded. After the third process, a process of selecting one containing a predetermined number of metal elements from the metal oxides selected in the third process may be additionally provided.

### Fourth Process

A Pourbaix diagram is created for each metal oxide selected in the third process, with a stable region being a pH and potential region in which the Gibbs free energy is 0.5 eV·atom⁻¹ or less.

### Fifth Process

Among Pourbaix diagrams created in the fourth process, a stable metal oxide that provides a Pourbaix diagram indicating the presence of a solid phase in a predetermined reaction (oxygen reduction reaction, etc.) is selected as an electrode catalyst. For example, when an acidic solution is used as an electrolytic solution, a metal oxide that is stable in a potential range corresponding to a pH range of the acidic solution in the Pourbaix diagram is selected. In addition, for example, when an alkaline solution is used as an electrolytic solution, a metal oxide that is stable in a potential range corresponding to a pH range of the alkaline solution in the Pourbaix diagram is selected.

Combinations of the metal oxides selected in the third process and the Pourbaix diagrams created in the fourth process may be recorded in a predetermined recording device, and a new metal oxide to be used as the electrode catalyst may be selected by searching the records in the recording device. The recording device may be an online search engine that allows searching in a network environment connected to the Internet.

According to the above data mining method, as a metal oxide that is stable in the oxygen reduction reaction (ORR) under acidic conditions, it is possible to search for not only generally known single-component metal oxides (compounds of one metal element and oxygen) but also multi-component metal oxides (compounds of a plurality of metal elements and oxygen). Examples of single-component metal oxides to be searched for include Sb₂O and W₂O₃. In addition, examples of multi-component metal oxides to be searched for include Hg(SbO₃)₂, Cd(SbO₃)₂, Zn(SbO₃)₂, Mn(SbO₃)₂, Ni(SbO₃)₂, Co(SbO₃)₂, Fe(SbO₃)₂, Sc₂(MoO₄)₃, Fe₂(MoO₄)₃, GaSbO₄, BiSbO₄, CsSbWO₆, RbSbWO₆, Ge₃Sb₂O₉, MgCr₂O₄, Sb₂WO₆, TiSnO₃, RbSbWO₆, and Li₄CrFe₃O₈.

As the single-component metal oxide, a metal oxide (Sb₂O, etc.) containing Sb is excellent as an electrode catalyst involved in an oxygen evolution reaction and an oxygen reduction reaction. In addition, W has the second highest stability after Sb, and as the multi-component metal oxide, a metal oxide containing Sb and W (Sb₂WO₆, etc.) is excellent as an electrode catalyst that accelerates an oxygen evolution reaction and an oxygen reduction reaction.

As described above, the data mining method of the present embodiment is a method of searching for a metal oxide without considering the surface state of the catalyst layer. Therefore, the data mining method of the present embodiment identifies Sb₂WO₆ that is stable only under acidic conditions. Here, in cases where the surface state and microkinetic modeling are considered, it is thought that it is possible to search for a metal oxide that is stable under alkaline conditions. According to the data mining method of the present embodiment, it is possible to easily and accurately search for metal oxides that function as an electrode catalyst in a predetermined reaction. In the fourth process, for each metal oxide, a pH and potential region in which the Gibbs free energy is 0.5 eV·atom⁻¹ or less is identified. Thereby, it is possible to identify metal oxides that are thermodynamically stable and suitable for a predetermined reaction with high accuracy.

### EXAMPLES

Hereinafter, the effects of the present invention will be more clearly understood with reference to examples. Here, the present invention is not limited to the following examples, and can be appropriately modified and implemented without departing from the spirit and scope of the invention.

### Data Mining

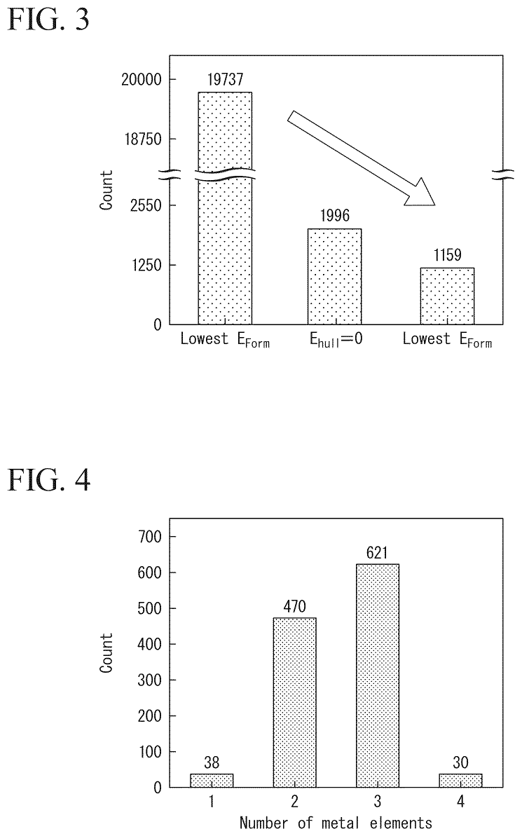

The data mining method of the above embodiment was performed on many types of metal oxides recorded in the database. FIG. 3 is a graph showing the numbers of metal oxides selected after the first process, after the second process, and after the third process. By the third process, the number of predetermined metal oxide candidates to be selected was narrowed to 1159.

FIG. 4 is a graph showing the 1,159 selected metal oxides classified according to the number of metal elements that constitute them. Among the 1,159 selected metal oxides, metal oxides containing two metal elements and metal oxides containing three metal elements are particularly abundant. Among the found metal oxides, Sb₂WO₆ was evaluated.

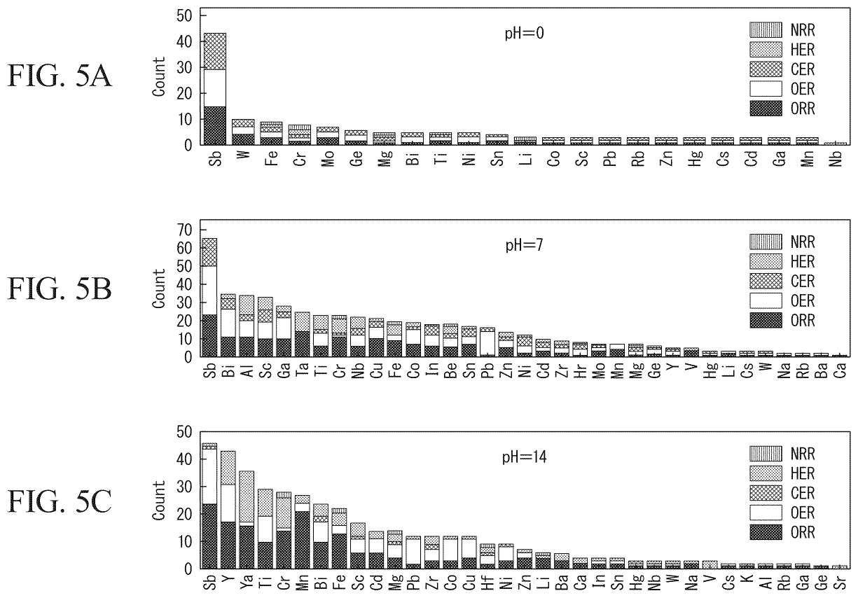

FIGS. 5A, 5B, and 5C are graphs comparing the thermodynamic stability of metal elements in reactions (nitrogen reduction reaction (NRR), hydrogen evolution reaction (HER), chlorine evolution reaction (CER), oxygen evolution reaction (OER), and oxygen reduction reaction (ORR)) in aqueous solutions with a pH of 0, 7, and 14. Regardless of the state of the aqueous solution, Sb has the best stability in the OER and ORR.

Based on these results, it was found that, as a single-component metal oxide, a metal oxide containing Sb (Sb₂O) was excellent as an electrode catalyst involved in an oxygen evolution reaction and an oxygen reduction reaction. In addition, it was found that, since W had the second highest stability after Sb, as a multi-component metal oxide, a metal oxide containing Sb and W was excellent as the electrode catalyst.

### Synthesis of Sb₂WO₆

Nanoplates of Sb₂WO₆ (an average thickness of 20 nm) found by the data mining method were synthesized by a hydrothermal synthesis method. Specifically, Sb₂WO₆ was synthesized by the following procedure. SbCl₃ powder (137 mg) and Na₂WO₄/2H₂O (99 mg) were dissolved in deionized water (15 mL) and stirred, a W solution was added, and the mixture was additionally stirred. The stirred solution was placed in a Teflon (registered trademark) autoclave (45 mL), and heated at 180° C. for 12 hours. In addition, the sample was centrifuged, washed with deionized water, and finally dried at 70° C. overnight to obtain Sb₂WO₆ nanoplates.

### Evaluation of Sb₂WO₆

X-ray diffraction (XRD) measurement was performed on the Sb₂WO₆. FIG. 6 is a graph showing the measurement results. The diffraction peak positions of the synthesized Sb₂WO₆ were almost the same as the diffraction peak positions of standard Sb₂WO₆. Based on the results, it was found that the synthesized Sb₂WO₆ had the same orthorhombic crystal structure as standard Sb₂WO₆.

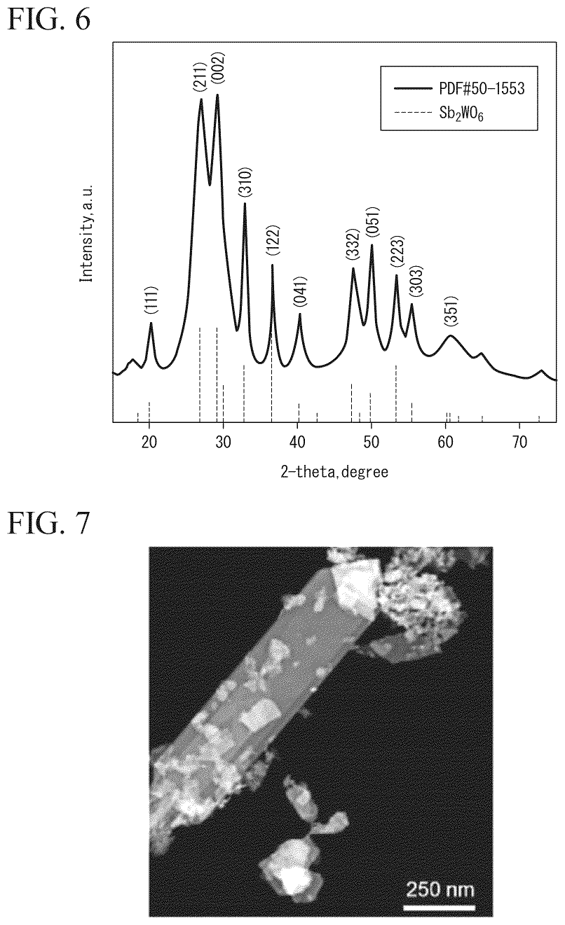

An image of the synthesized Sb₂WO₆ was obtained using a high-angle annular dark-field scanning transmission electron microscope (HAADF-STEM). FIG. 7 shows the obtained image. From the image, the synthesized Sb₂WO₆ can be observed.

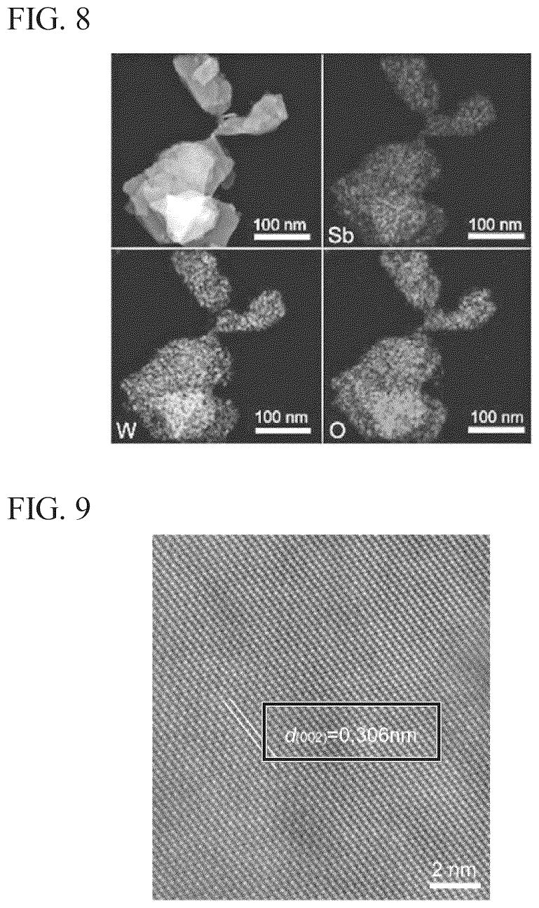

The synthesized Sb₂WO₆ was subjected to elemental mapping by energy dispersive X-ray (EDX) spectroscopy. FIG. 8 shows images of the elemental mapping results. It was found that the element ratio Sb:W:O was 2.00:0.99:6.04, which was almost the same as the theoretical stoichiometric value.

The synthesized Sb₂WO₆ was subjected to ICP-OES measurement. FIG. 9 is an image showing the measurement results. From the image, lattice fringes arranged at intervals could be confirmed on the (002) plane of the orthorhombic crystal structure.

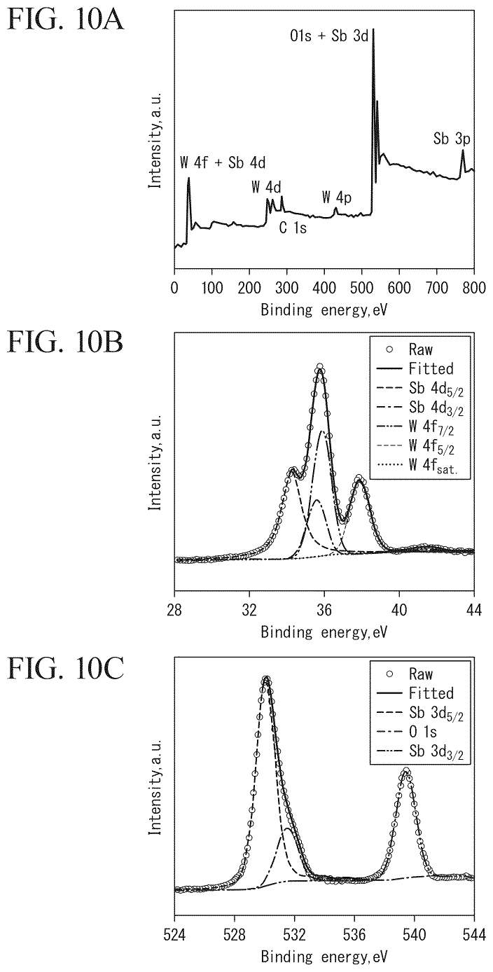

The synthesized Sb₂WO₆ was subjected to X-ray photoelectron spectroscopy (XPS) measurement. FIG. 10A is a graph showing the spectrum across the entire range of photoelectron energy obtained by measurement. Peaks corresponding to electrostatic orbitals of Sb, W, and O could be confirmed. FIG. 10B is an enlarged view of the spectrum of FIG. 10A, showing peak parts of W in the 4f orbital and Sb in the 4d orbital. FIG. 10C is an enlarged view of the spectrum of FIG. 10A, showing peak parts of O in the 1s orbital and Sb in the 3d orbital.

An oxygen reduction reaction was caused using a rotating ring-disk electrode method (RRDE) in an 1 M HClO₄ (pH=1.3) acidic solution and an 0.1 M KOH (pH=12.6) alkaline solution. A catalyst layer containing Sb₂WO₆ was provided on the disk electrode (carbon black substrate), and an oxygen reduction reaction was caused through the catalyst layer. Here, the disk electrode corresponded to the first electrode, and the ring electrode corresponded to the second electrode.

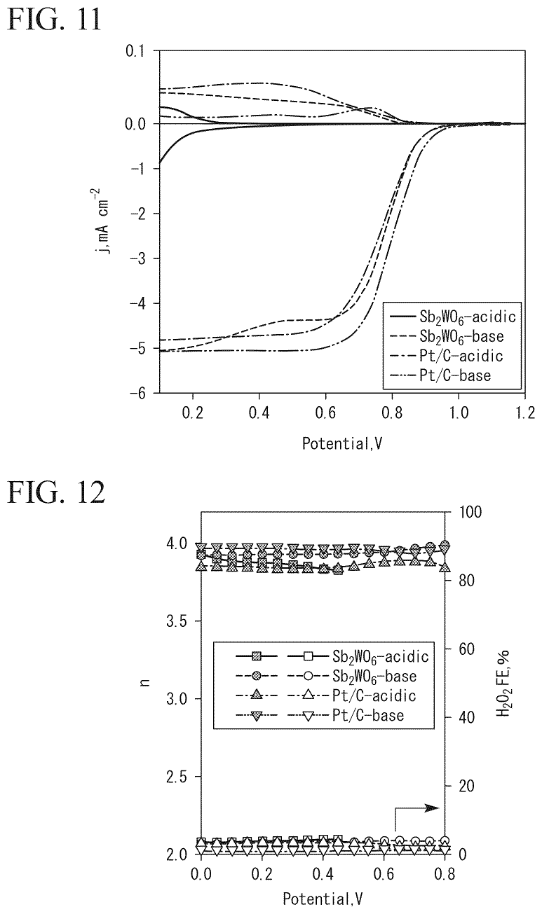

FIG. 11 is a graph showing the LSV curves obtained in this case. With the reversible hydrogen electrode V_{RHE}, the onset potential was 0.94 V, the half-wave potential was 0.78 V, and a disk current density of 0.05 mA/cm² was obtained. These properties were as excellent as those when a catalyst layer containing commercially available Pt and C was used.

In the oxygen reduction reaction using the rotating ring-disk electrode method, the number of electrons contributing to the reaction was measured. FIG. 12 is a graph showing the measurement results. It was found that, in both an acidic solution and an alkaline solution, the four-electron oxygen reduction reaction was maintained, and almost no H₂O₂ was produced. The high current density shown in FIG. 11 was thought to be due to the four-electron reaction.

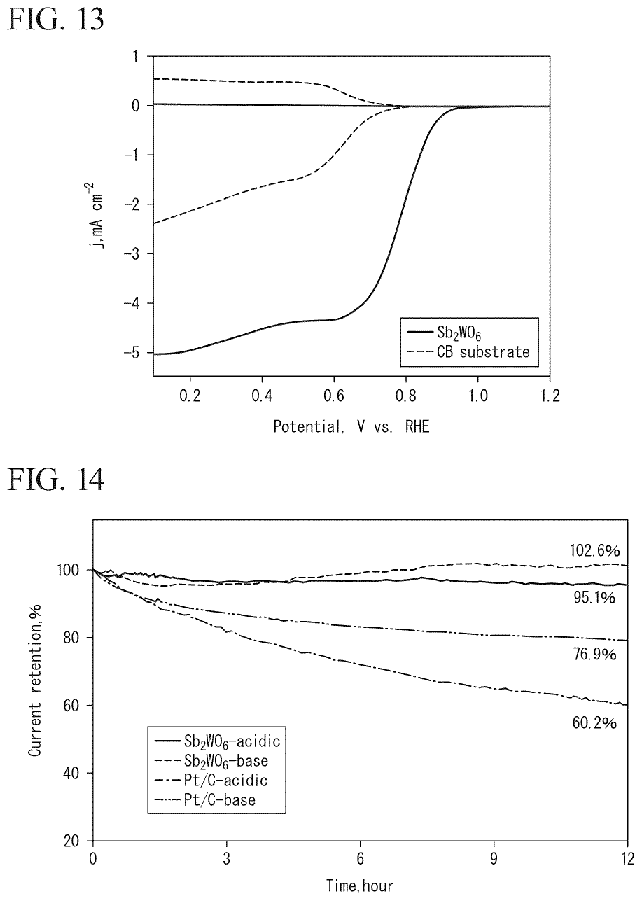

When no catalyst layer was formed on the disk electrode, an oxygen reduction reaction was caused without the catalyst layer therebetween, and the same measurement was performed. FIG. 13 is a graph comparing the current density obtained by the oxygen reduction reaction when a catalyst layer containing Sb₂WO₆ was inserted and when a catalyst layer containing Sb₂WO₆ was not inserted. The current density when a catalyst layer was inserted was 2 to 5 times the current density when no catalyst layer was inserted. The effect of increasing the current density observed when comparing the two current densities was thought to be due to the oxygen reduction reaction involving four electrons rather than the effect of Sb₂WO₆.

In an acidic solution and an alkaline solution, Sb₂WO₆ was mounted on a carbon cloth electrode as a catalyst, and chronoamperometry measurement was performed. FIG. 14 is a graph showing the measurement results. The currents flowing continuously in an acidic solution and an alkaline solution were 95.1% and 102.6% of the initial current after 12 hours, respectively, and in both cases, a higher retention rate was exhibited compared to that of commercially available catalysts containing Pt and C. This is thought to be because Sb and W contained in the catalyst layer had high chemical stability.

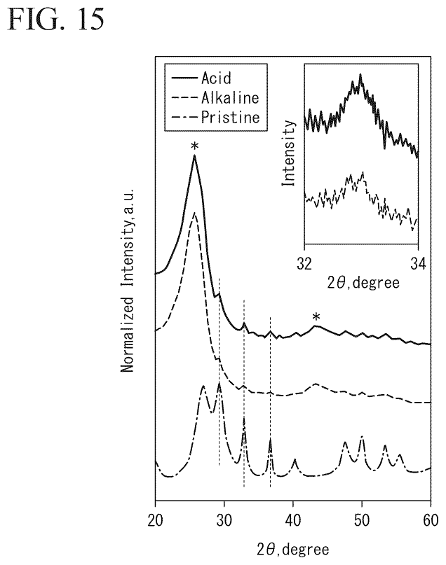

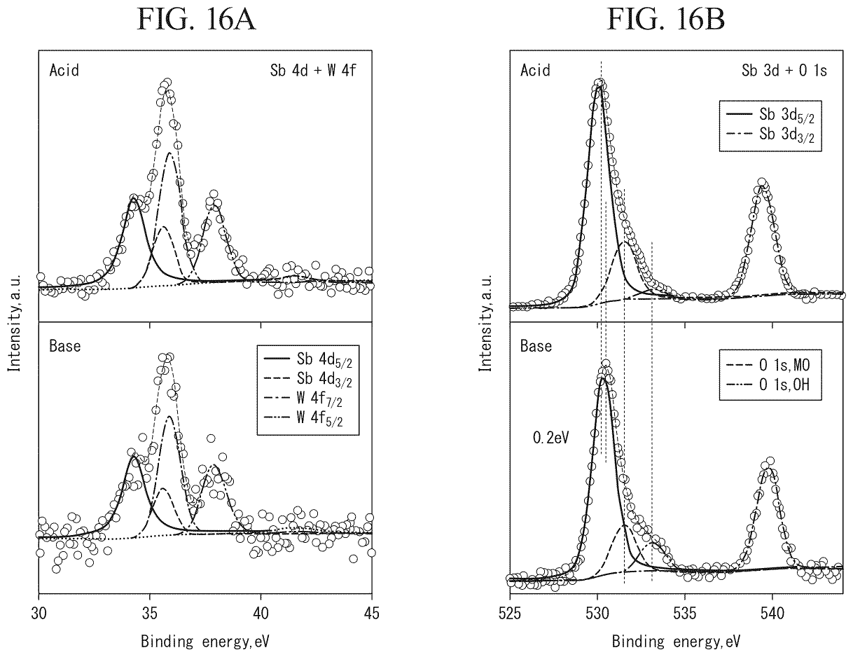

The results of X-ray diffraction measurement and X-ray electron spectroscopy measurement of Sb₂WO₆ obtained immediately after stability evaluation were corrected using a density functional theory (DFT) to reflect the crystal structure of Sb₂WO₆ when placed in an acidic electrolytic solution and an alkaline electrolytic solution. FIG. 15 is a graph showing the X-ray diffraction measurement results in FIG. 6 before and after correction. FIGS. 16A and 16B are graphs showing the X-ray photoelectron spectroscopy measurement results in FIGS. 10B and 10C before and after correction, respectively.

In the graph in FIG. 15, in the case of Sb₂WO₆ (Pristine) before correction, strong peaks were observed at diffraction angles of 25 degrees and 42 degrees, but in the case of Sb₂WO₆ (Acid, Alkaline) after correction, these peaks disappeared. The half-width of the peak in the acidic electrolytic solution was 20 to 30% of the half-width of the peak in the alkaline electrolytic solution. These results could be confirmed from the Raman spectrum measurement results.

The peak of Sb in the 4d orbital in the spectrum of FIG. 16A was blue-shifted by about 0.1 eV with respect to the same peak in the spectrum of FIG. 10B. In addition, the peak of Sb in the 3d orbital in the spectrum of FIG. 16B was blue-shifted by about 0.2 eV with respect to the same peak in the spectrum of FIG. 10B.

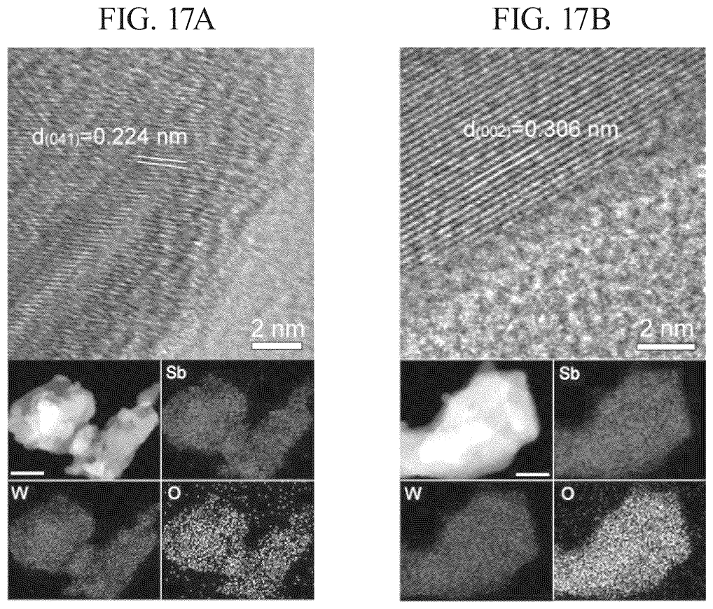

FIG. 17A shows a TEM image of Sb₂WO₆ in an acidic electrolytic solution (top) and an EDS elemental mapping image (bottom). In the Sb₂WO₆, the (041) plane extended from the inside toward the surface, and almost no structural change was observed.

FIG. 17B shows a TEM image of Sb₂WO₆ in an alkaline electrolytic solution (top) and an EDS elemental mapping image (bottom). A reduced and less ordered structure was observed in the surface layer of the Sb₂WO₆.

Comparing the EDS elemental mapping images, in both cases, Sb, W, and O were uniformly distributed, but the ratio of Sb to W (Sb/W) differed between the acidic electrolytic solution and the alkaline electrolytic solution. The ratio of Sb to W remained at 2.00±0.02 in the acidic electrolytic solution immediately after stability evaluation, but increased to 2.03±0.02 in the alkaline electrolytic solution immediately after stability evaluation.

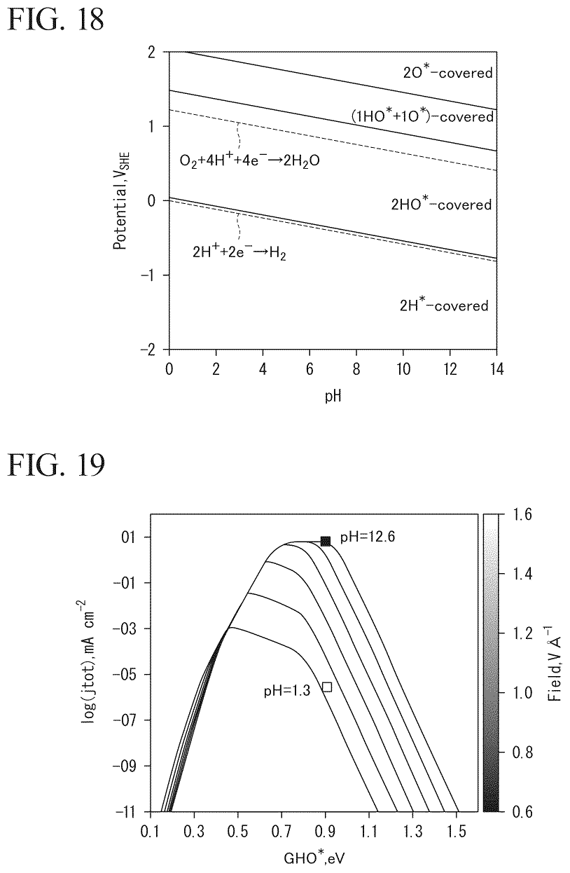

FIG. 18 is a Pourbaix diagram showing the surface state of a catalyst layer containing Sb₂WO₆. When pH=0 and V>0, the surface of the catalyst layer was stable when covered with oxygen. When pH=0 and V<0, the surface of the catalyst layer was stable when covered with hydrogen. The potential window (region between two broken lines) was about 1.5 V.

The obtained current density was measured, which corresponded to the binding energy of hydroxide ions to the catalyst layer when the pH around the catalyst layer was in a range of 1.3 or more and 12.6 or less. FIG. 19 is a graph showing the results. Volcano-shaped plots are shown according to the Sabatier principle. That is, there was a current density peak for each pH, and the magnitude of the current density was proportional to the pH.

### EXPLANATION OF REFERENCES

100 Fuel cell
101 First electrode
102 Second electrode
103 First catalyst layer
104 Second catalyst layer
105 Electrolyte
106 Container
107 Wiring
108 Power generation unit
109 Oxygen supply source
110 Reducing agent supply source

What is claimed is:

1. An oxygen reduction catalyst comprising Sb₂WO₆.

2. An electrolysis device, comprising:
a first electrode;
a first catalyst layer provided on the first electrode;
a second electrode;
a second catalyst layer provided on the second electrode;
an electrolyte interposed between the first electrode and the second electrode; and
a container containing the first electrode, the first catalyst layer, the second electrode, the second catalyst layer, and the electrolyte,
wherein the first catalyst layer contains Sb₂WO₆.

3. A catalyst layer that is provided in an electrode constituting an electrolysis device and promotes an electrolytic reaction, comprising Sb₂WO₆.

4. An electrolysis method, comprising:
a process of generating a potential difference between two electrodes with an electrolyte therebetween;
a process of supplying oxygen molecules to a first electrode on the high potential side between the electrodes; and
a process of supplying a reducing agent for the oxygen molecules to a second electrode on the low potential side between the electrodes,
wherein a catalyst layer containing Sb₂WO₆ is provided on the surface of the first electrode.

5. The electrolysis method according to claim 4, wherein an oxygen reduction reaction is caused in the catalyst layer to generate water.

6. The electrolysis method according to claim 4, wherein hydrogen molecules or hydrogen compound molecules are used as the reducing agent.

7. The electrolysis method according to claim 4,
wherein the pH of the electrolyte around the catalyst layer and the potential of the first electrode are adjusted so that the values are included in a region between a first line segment and a second line segment in a Pourbaix diagram,
the first line segment is a line segment connecting a point at which the pH is 0 and the potential is 0.6 V and a point at which the pH is 14 and the potential is −0.226 V, and
the second line segment is a line segment connecting a point at which the pH is 0 and the potential is 1 V and a point at which the pH is 14 and the potential is 0.174 V.

8. The electrolysis method according to claim 4, wherein the pH around the catalyst layer is 1.3 or more and 12.6 or less.

9. A data mining method of searching for a metal oxide as a catalyst in an oxygen reduction reaction, comprising:
a first process of selecting a predetermined metal oxide from metal oxides recorded in a database;
a second process of selecting a metal oxide that satisfies the condition that Eₕᵤₗₗ is 0 from the metal oxides selected in the first process;
a third process of selecting a metal oxide that satisfies the condition that E_{Form} is minimized from the metal oxides selected in the second process;
a fourth process of creating a Pourbaix diagram for each metal oxide selected in the third process, with a stable region being a pH and potential region in which the Gibbs free energy is 0.5 eV·atom⁻¹ or less; and
a fifth process of selecting, as an electrode catalyst, a metal oxide that provides a Pourbaix diagram indicating the presence of a solid phase in a predetermined reaction among the Pourbaix diagrams created in the fourth process.

10. The data mining method according to claim 9, wherein combinations of the metal oxides selected in the third process and the Pourbaix diagrams created in the fourth process are recorded in a predetermined recording device, and the metal oxide to be used as the electrode catalyst is selected by searching records in the recording device.

11. The data mining method according to claim 9, further comprising a process of selecting one containing a predetermined number of metal elements from the metal oxides selected in the third process.
